(19) United States
(12) Patent Application Publication
(10) Pub. No.: US 2023/0404653 A1
(43) Pub. Date: Dec. 21, 2023

(54) HAND-PIECE FOR AN ELECTROSURGICAL INSTRUMENT

(71) Applicant: GYRUS MEDICAL LIMITED, Cardiff (GB)

(72) Inventor: Matthew RUNNALLS, Cardiff (GB)

(73) Assignee: GYRUS MEDICAL LIMITED, Cardiff (GB)

(21) Appl. No.: 18/212,242

(22) Filed: Jun. 21, 2023

Related U.S. Application Data
(60) Provisional application No. 63/353,921, filed on Jun. 21, 2022.

(51) Int. Cl. A61B 18/14

(52) U.S. Cl. CPC A61B 18/1482; A61B 2018/00589

(57) ABSTRACT

The present disclosure seeks to address the problem of carbon tracking in electrosurgical instruments caused by a build-up of saline by providing the hand-piece of the instrument with a drainage system, for example, in the form of a lumen leading to an opening in the outer casing of the hand-piece. Any trapped saline between the sealing surfaces thereby drains from the cavity within the hand-piece, ensuring that an air-gap is formed between the contacts, and thus preventing the possibility of carbon tracking between the contacts.

### TECHNICAL FIELD

The present invention relates to electrosurgical instruments. More specifically, the present invention relates to a hand-piece for an electrosurgical instrument.

### BACKGROUND TO THE INVENTION AND PRIOR ART

Surgical instruments, including radio frequency (RF) electrosurgical instruments, have become widely used in surgical procedures where access to the surgical site is restricted to a narrow passage, for example, in minimally invasive “keyhole” surgeries.

RF electrosurgical instruments typically include an active electrode which forms a distal RF tip of the instrument, and a return electrode. The distal RF tip provides tissue ablation and/or coagulation effects at a surgical site when a RF power signal is delivered to the electrodes. Some RF instruments also implement a mechanical shaver, with the main mechanical shaving componentry located on the opposite side of the distal tip.

Within the hand-piece of such instruments, sealing surfaces are used to maintain a separation between the active and return mating contacts, which act to prevent the shorting between the active and return contacts that occurs if saline becomes trapped in the connection.

However, if saline becomes trapped or pools between the active and return contacts, the hand-piece or instrument material can become susceptible to carbon tracking when a high voltage difference is applied to the contacts.

### SUMMARY OF THE INVENTION

In known RF electrosurgical instruments that implement an active electrode and a return electrode, the hand-piece typically implements sealing surfaces to maintain a separation between the active and return mating contacts, which act to prevent the shorting between the active and return contacts that occurs if saline becomes trapped in the connection. However, if saline becomes trapped between the active and return contacts, carbon tracking can occur when a high voltage difference is applied. The carbon tracking occurs as a result of the saline on the surface of the material and leakage current across the sealing surface. The present disclosure therefore seeks to address this problem by providing the hand-piece with a drainage system, for example, in the form of a lumen leading to an opening in the outer casing of the hand-piece. Any trapped saline between the sealing surfaces thereby drains from the cavity within the hand-piece, ensuring that an air-gap is formed between the contacts, and thus preventing the possibility of carbon tracking between the contacts, which can cause an electrical short.

A first aspect of the present invention provides a hand-piece for an electrosurgical instrument, comprising an inner moulding comprising a first set of electrical contacts configured to be connected to at least two electrodes, and an outer casing formed around the inner moulding such that a cavity is formed therebetween, the outer casing comprising a second set of electrical contacts configured to be connected to an RF energy supply, wherein the second set of electrical contacts are arranged so as to mate with the first set of electrical contacts within the cavity, and at least one lumen extending between a first opening on an inner wall of the outer casing and a second opening on an outer wall of the outer casing, for transporting fluid from the cavity.

As such, a channel is provided for draining any fluid, such as saline, that has built up in the cavity where the electrical connections for connecting the electrodes to the energy supply are located. In doing so, this prevents the fluid from pooling in the hand-piece and causing carbon tracking to occur between the electrical contacts.

The hand-piece may further comprise a plurality of sealing surfaces within the cavity. More specifically, at least one sealing surface may be provided between two pairs of mating electrical contacts. These sealing surfaces thus act to maintain a first layer of separation between the electrical contacts to stop fluid within the cavity causing an electrical short. More preferably, two sealing surfaces are provided between two pairs of mating electrical contacts, thus providing two barriers between the electrical contacts.

In some arrangements, at least one lumen is provided between two sealing surfaces, for example, the two sealing surfaces provided between the two pairs of mating electrical contacts. As such, fluid is prevented from pooling between the two sealing surfaces, which in turn prevents the occurrence of carbon tracking.

At least one lumen may also be provided between a sealing surface and a pair of mating electrical contacts. In doing so, this creates dry areas around the electrical contacts for each electrode.

The plurality of sealing surfaces may comprise a plurality of O-rings. In such cases, the inner wall of the outer casing and an outer wall of the inner moulding may comprise a plurality of notches for receiving the plurality of O-rings. In doing so, the O-rings are held in place and prevented from sliding laterally within the cavity, which could result in leakage between the O-rings.

The first and second set of electrical contacts are preferably configured to provide respective pairs of mating electrical contacts. That is to say, for each electrical contact on the inner moulding, a corresponding electrical contact is provided on the outer casing.

For example, the first set of electrical contacts may comprise a first electrical contact and a second electrical contact, and the second set of electrical contacts may comprise a third electrical contact and a fourth electrical contact, wherein the first and third electrical contacts are arranged to form a first pair of mating electrical contacts, and the second and fourth electrical contacts are arranged to form a second pair of mating electrical contacts.

In such cases, the first pair of mating electrical contacts may be configured to connect a first electrode to the RF energy supply, and the second pair of mating electrical contacts may be configured to connect a second electrode to the RF energy supply. For example, the first electrode may be an active electrode and the second electrode may be a return electrode.

In some arrangements, the hand-piece further comprises a suction tube connected to the lumen at the second opening for suctioning fluid from the cavity. Alternatively, no suction tube is provided, and the fluid is allowed to exit the hand-piece to the external environment.

A further aspect of the present invention provides an electrosurgical instrument, comprising a hand-piece as described above, an elongate shaft extending from a distal end of the hand-piece, and an end effector positioned at a distal end of the elongate shaft, the end effector comprising an active electrode and a return electrode, wherein the elongate shaft comprises means for connecting the active electrode and the return electrode to the first set of electrical contacts.

Another aspect of the present invention provides an electrosurgical system, comprising an RF electrosurgical generator, and an electrosurgical instrument as described above, the arrangement being such that in use the RF electrosurgical generator supplies an RF coagulation or ablation signal via the first and second sets of electrical contacts to the active electrode and the return electrode.

Yet a further aspect of the present invention may provide a method for processing an instrument for surgery, the method comprising obtaining an electrosurgical instrument and/or end effector described herein, sterilizing the electrosurgical instrument and/or end effector, and storing the electrosurgical instrument and/or end effector in a sterile container.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 shows an example of the electrosurgical instrument system comprising an RF electrosurgical instrument according to the present disclosure;

FIG. 2 illustrates an example end effector of an electrosurgical instrument according to the present disclosure;

FIG. 3 illustrates a hand-piece in accordance with the present disclosure.

### DETAILED DESCRIPTION OF THE EMBODIMENTS

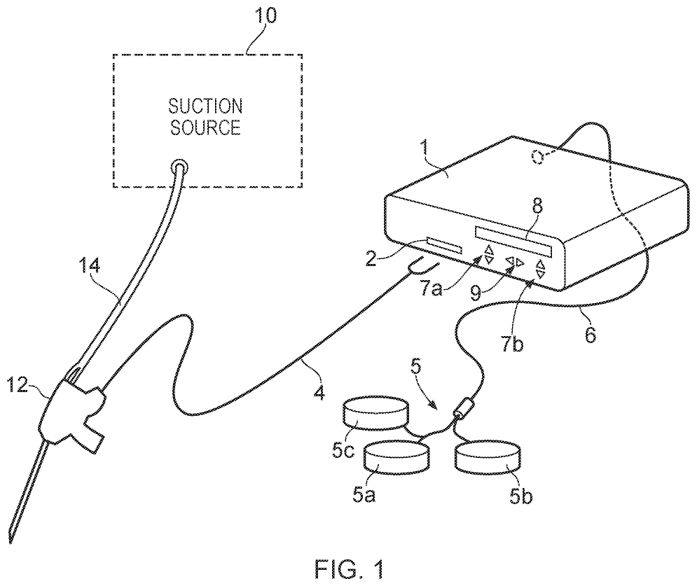

FIG. 1 shows an electrosurgical apparatus including an electrosurgical generator 1 having an output socket 2 that provides a radio frequency (RF) output (e.g. a RF power signal), via a connection cord 4, to an electrosurgical instrument 3 having an end effector that may be configured to provide a mechanical shaving function, as well as a electrosurgical cutting and coagulation functions. The instrument 3 has a suction tube 14 which is connected to a suction source 10. Activation of the generator 1 may be performed from the instrument 3 via a handswitch 12a on the hand-piece 12 of the instrument 3, or by means of a footswitch unit 5 connected separately to the rear of the generator 1 by a footswitch connection cord 6. In the illustrated embodiment, the footswitch unit 5 has three footswitches 5a, 5b and 5c for selecting a mechanical shaving mode, a coagulation mode, or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between different modes.

As described above, the electrosurgical instrument 3 may be a dual sided (or an opposite sided) RF shaver device. In this respect, the main RF componentry and the manual shaving/cutting componentry of the instrument 3 can be provided on opposite sides of a distal end portion of the instrument 3. However, it will also be appreciated that the electrosurgical instrument 3 may be an RF device providing only electrosurgical cutting and coagulation functions.

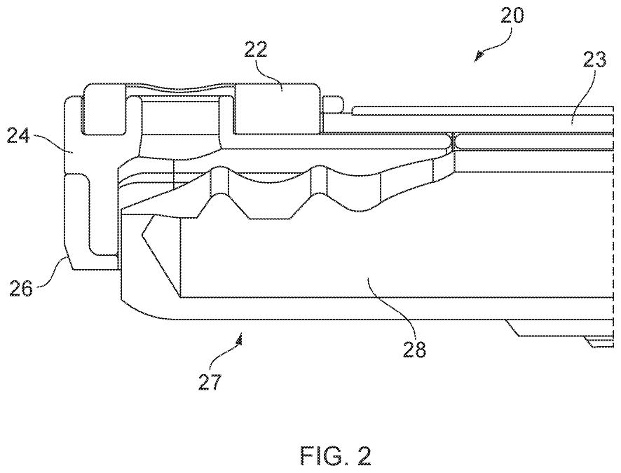

An example of an end effector having an RF shaver distal tip 20 is illustrated by FIG. 2, wherein the distal tip 20 comprises an active electrode 32 for tissue treatment (also referred to as the “active tip”), an insulating casing 24 arranged to receive the active electrode 22 and a return electrode 26. The insulating casing 24 is provided between the active electrode 22 and the return electrode component 26 to physically separate those components, and may be formed from any suitable material, such as ceramic or a polymer. The active electrode 22 and return electrode 26 may be formed from any suitable metal, for example, copper, stainless steel, tungsten or an alloy of tungsten and platinum.

In this example, the return electrode 26 acts as an outer shaft that houses a rotating inner blade 28 therein, which provides the mechanical shaving function. In this respect, the return electrode 26 comprises an opening 27 that acts as a window to the rotating inner blade 28. However, as mentioned above, it will of course be appreciated that the end effector may also be provided without the opening 27 and rotating inner blade 28, providing only electrosurgical cutting and coagulation functions.

To deliver the RF power signal to the active electrode 22, an electrical conductor 23 will be provided that extends along the shaft of the instrument 3 to the hand piece 12 for connection to the generator 1. Likewise, a return path (not shown) is also provided by some suitable conductive means that extends from the return electrode 26 to the hand-piece 12.

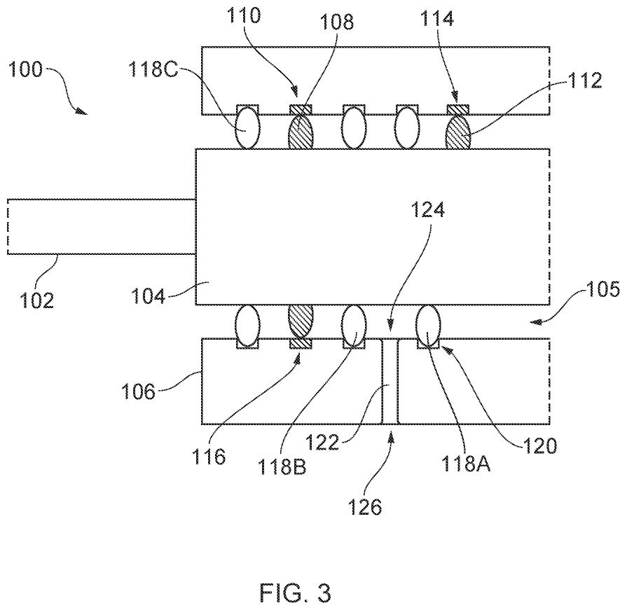

An example of the hand-piece 12 according to the present disclosure is illustrated by FIG. 3, which shows a cross-sectional view of the distal end 100 of the hand-piece 12. As can be seen from FIG. 3, the shaft 102 of the instrument 3 containing the electrical conductor for the active electrode 22 and the return path for the return electrode 26 is coupled to an inner moulding 104 configured to house the electrical and mechanical connections for the end effector. That is to say, it contains the connections for connecting the electrodes to the RF power supply, and if applicable, for connecting the mechanical shaver to drive componentry in order to drive the rotating inner blade 28. The hand-piece 12 further comprises an outer casing 106 disposed around the outside of the inner moulding 104. The outer casing 106 is configured to house the electrical and mechanical connections leading back to the generator 1.

In order to connect the active and return electrodes to the RF power supply, electrical contacts extend from the outer wall of the inner moulding 104 that are configured to mate with a corresponding set of contacts on the internal wall of the outer casing 106, the respective sets of contacts mating within a cavity 105 formed between the inner moulding 104 and the outer casing 106. For the active electrode 22, a first set of contacts are provided comprising a first contact 108 extending from the inner moulding 104 and a corresponding second contact 110 on the outer casing 106. In this respect, the first contact 108 is connected to the electrical conductor 23 that leads up to the active electrode 22, whilst the second contact 110 is connected to an electrical conductor (not shown) leading back to the generator 1. Similarly, for the return electrode 26, a second set of contacts are providing comprising a first contact 112 extending from the inner moulding 104 and a corresponding second contact 114 on the outer casing 106. Again, the first contact 112 is connected to the return path of the return electrode 26, whilst the second contact 114 is connected to an electrical conductor (not shown) leading back to the generator 1. It will also be appreciated, as shown by FIG. 3, that further sets of contacts 116 may also be provided for connecting other electrical components of the instrument.

A plurality of sealing surfaces 118A-C are provided between the inner moulding 104 and outer casing 106 for maintaining a separation between the electrical contacts 108, 110, 112, 114, 116 of the hand-piece 12. In this example, the sealing surfaces 118A-C are provided in the form of O-rings, made from any suitable elastomeric or rubber material such as Silicone rubber, although it will be appreciated that any suitable means may be used that is capable of providing a sealed separation between the electrical contacts 108, 110, 112, 114, 116. The outer casing 106 comprises notches 120 within the inner wall for receiving the O-rings 118A-C to ensure that the O-rings are retained in position and do not slide laterally within the cavity 105 between the inner moulding 104 and outer casing 106.

As shown in FIG. 3, two O-rings 118A-B are arranged between the first set of contacts 108, 110 for the active electrode and the second set of contacts 112, 114 for the return electrode, which acts to prevent shorting between the active and return electrodes when saline becomes trapped in the cavity 105. To prevent saline building up between the O-rings 118A-B, a drainage system is provided that comprises at least one drainage lumen 122 extending through the outer casing 106. The lumen 122 extends from a first opening 124 in the inner wall of the outer casing 106 positioned between the two O-rings 118A-B and a second opening 126 in the outer wall of the casing 106. The second opening 126 may be connected to a suction tube or similar (not shown), or no suction tube may be provided and the saline may be allowed to simply drain from the hand-piece 12. As such, an air-gap between the O-rings 118A-B is formed as the saline drains from the cavity 105, thus preventing the possibility of carbon tracking between the two sets of contacts 108, 110, 112, 114.

Whilst one drainage lumen 122 is shown, it will of course be appreciated that a second lumen may also be provided, for example, at different circumferential positions on the hand-piece 12. Similarly, whilst the drainage lumen 122 is shown as being positioned between the two O-rings 118A-B, it will be appreciated that a drainage lumen may be provided between one sealing surface and one set of contacts, which may be provided in addition to or instead of the drainage lumen 122 as shown in FIG. 3. For example, a drainage lumen may be provided between the first O-ring 118A and the contacts 112, 114 for the return electrode and/or a drainage lumen may be provided between the second O-ring 118B and the contacts 108, 110 for the active electrode, thereby providing dry areas around either sets of contacts.

Various further modifications to the above-described embodiments, whether by way of addition, deletion or substitution, will be apparent to the skilled person to provide additional embodiments, any and all of which are intended to be encompassed by the appended claims.

The electrosurgical instrument and/or end effector used in conjunction with the hand-piece disclosed herein can be designed to be disposed of after a single use, or they can be designed to be used multiple times. In either case, however, the electrosurgical instrument and/or end effector can be reconditioned for reuse after at least one use. Reconditioning can include a combination of the steps of disassembly of the electrosurgical instrument, followed by cleaning or replacement of particular pieces, and subsequent reassembly. In particular, the electrosurgical instrument can be disassembled, and any number of particular pieces or parts of the device (such as the end effector) can be selectively replaced or removed in any combination. Upon cleaning and/or replacement of particular parts, the electrosurgical instrument can be reassembled for subsequent use either at a reconditioning facility, or by a surgical team immediately prior to a surgical procedure. Those of ordinary skill in the art will appreciate that the reconditioning of an electrosurgical instrument can utilize a variety of different techniques for disassembly, cleaning/replacement, and reassembly. Use of such techniques, and the resulting reconditioned electrosurgical instrument, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The electrosurgical instrument may also be sterilized using any other technique known in the art, including but limited to beta or gamma radiation, ethylene oxide, or steam.

1. A hand-piece for an electrosurgical instrument, comprising:
an inner moulding comprising a first set of electrical contacts configured to be connected to at least two electrodes; and
an outer casing formed around the inner moulding such that a cavity is formed therebetween, the outer casing comprising:
a second set of electrical contacts configured to be connected to an RF energy supply, wherein the second set of electrical contacts are arranged so as to mate with the first set of electrical contacts within the cavity; and
at least one lumen extending between a first opening on an inner wall of the outer casing and a second opening on an outer wall of the outer casing, for transporting fluid from the cavity.

2. A hand-piece according to claim 1, further comprising a plurality of sealing surfaces within the cavity.

3. A hand-piece according to claim 2, wherein at least one sealing surface is provided between two pairs of mating electrical contacts.

4. A hand-piece according to claim 3, wherein two sealing surfaces are provided between two pairs of mating electrical contacts.

5. A hand-piece according to claim 2, wherein at least one lumen is provided between two sealing surfaces.

6. A hand-piece according to claim 2, wherein at least one lumen is provided between a sealing surface and a pair of mating electrical contacts.

7. A hand-piece according to claim 2, wherein the plurality of sealing surfaces comprises a plurality of O-rings.

8. A hand-piece according to claim 7, wherein the inner wall of the outer casing and an outer wall of the inner moulding comprise a plurality of notches for receiving the plurality of O-rings.

9. A hand-piece according to claim 1, wherein the first and second set of electrical contacts are configured to provide respective pairs of mating electrical contacts.

10. A hand-piece according to claim 1, wherein the first set of electrical contacts comprises a first electrical contact and a second electrical contact, and the second set of electrical contacts comprises a third electrical contact and a fourth electrical contact, wherein the first and third electrical contacts are arranged to form a first pair of mating electrical contacts, and the second and fourth electrical contacts are arranged to form a second pair of mating electrical contacts.

11. A hand-piece according to claim 10, wherein the first pair of mating electrical contacts are configured to connect a first electrode to the RF energy supply, and the second pair of mating electrical contacts are configured to connect a second electrode to the RF energy supply.

12. A hand-piece according to claim 11, wherein the first electrode is an active electrode and the second electrode is a return electrode.

13. A hand-piece according to claim 1, further comprising a suction tube connected to the lumen at the second opening for suctioning fluid from the cavity.

14. An electrosurgical instrument, comprising:
a hand-piece according to claim 1;
an elongate shaft extending from a distal end of the hand-piece; and
an end effector positioned at a distal end of the elongate shaft, the end effector comprising an active electrode and a return electrode, wherein the elongate shaft comprises means for connecting the active electrode and the return electrode to the first set of electrical contacts.

15. An electrosurgical system, comprising:
an RF electrosurgical generator; and
an electrosurgical instrument according to claim 14, the arrangement being such that in use the RF electrosurgical generator supplies an RF coagulation or ablation signal via the first and second sets of electrical contacts to the active electrode and the return electrode.
